(19) United States
(12) Patent Application Publication
(10) Pub. No.: US 2019/0043069 A1
(43) Pub. Date: Feb. 7, 2019

(54) SYSTEM AND METHOD FOR CONDUCTING ONLINE MARKET RESEARCH

(71) Applicant: NURALOGIX CORPORATION, Toronto (CA)

(72) Inventors: Kang LEE, Toronto (CA); Pu ZHENG, Toronto (CA)

(21) Appl. No.: 16/076,507

(22) PCT Filed: Feb. 8, 2017
(86) PCT No.: PCT/CA2017/050143

§ 371 Date: Aug. 8, 2018

Related U.S. Application Data
(60) Provisional application No. 62/292,583, filed on Feb. 8, 2016.

(51) Int. Cl. G06Q 30/02; G16H 50/20; G06F 3/01

(52) U.S. Cl. CPC G06Q 30/0203; G06F 3/013; G16H 50/20

(57) ABSTRACT

A method and system for conducting online market research is provided. Computer-readable instructions to a computing device of a participant, the computing device having a display, a network interface coupled to a network, and a camera configured to capture image sequences of a user of the computing device. The computer-readable instructions cause the computing device to simultaneously display at least one of an image, video, and text via the display and capture an image sequence of the participant via the camera, and transmit the captured image sequence to a server via the network interface. The image sequence is processed using an image processing unit to determine a set of bitplanes of a plurality of images in the captured image sequence that represent the hemoglobin concentration (HC) changes of the participant to detect the person's invisible emotional states based on HC changes. The image processing unit is trained using a training set comprising a set of subjects for which emotional state is known.

### TECHNICAL FIELD

The following relates generally to market research and more specifically to an image-capture based system and method for conducting online market research.

### BACKGROUND

Market research, such as via focus groups, has been employed as an important tool for acquiring feedback regarding new products, as well as various other topics.

A focus group may be conducted as an interview, conducted by a trained moderator among a small group of respondents. Participants are generally recruited on the basis of similar demographics, psychographics, buying attitudes, or behaviors. The interview is conducted in an informal and natural way where respondents are free to give views from any aspect. Focus groups are generally used in the early stages of product development in order to better plan a direction for a company. Focus groups enable companies that are exploring new packaging, a new brand name, a new marketing campaign, or a new product or service to receive feedback from a small, typically private group in order to determine if their proposed plan is sound and to adjust it if needed. Valuable information can be obtained from such focus groups and can enable a company to generate a forecast for its product or service.

Traditional focus groups can return good information, and can be less expensive than other forms of traditional marketing research. There can be significant costs however. Premises and moderators need to be provided for the meetings. If a product is to be marketed on a nationwide basis, it would be critical to gather respondents from various locales throughout the country since attitudes about a new product may vary due to geographical considerations. This would require a considerable expenditure in travel and lodging expenses. Additionally, the site of a traditional focus group may or may not be in a locale convenient to a specific client, so client representatives may have to incur travel and lodging expenses as well.

More automated focus group platforms have been introduced, but they are laboratory based and are generally able to test only a small group of consumers simultaneously with high costs. Further, except for a few highly specialized labs, most labs are only capable of measuring participants' verbalized subjective reports or ratings of consumer products under testing. However, studies have found that most people make decisions based on their inner emotions that are often beyond their conscious awareness and control. As a result, marketing research based on consumers' subjective reports often fails to reveal the genuine emotions on which consumers' purchasing decisions are based. This may be one reason why each year 80% of new products fail despite the fact that billions of dollars are spent on marketing research.

Electroencephalograms and functional magnetic resonance imaging can detect invisible emotions, but they are expensive and invasive and not appropriate for use with a large number of product testing participants who are all over the world.

### SUMMARY

In one aspect, a method for conducting online market research is provided, the method comprising: transmitting computer-readable instructions to a computing device of a participant, the computing device having a display, a network interface coupled to a network, and a camera configured to capture image sequences of a user of the computing device, the computer-readable instructions causing the computing device to simultaneously display at least one content item via the display and capture an image sequence of the participant via the camera, and transmit the captured image sequence to a server via the network interface; and processing the image sequence using a processing unit configured to determine a set of bitplanes of a plurality of images in the captured image sequence that represent the hemoglobin concentration (HC) changes of the participant, detect the participant's invisible emotional states based on the HC changes, and output the detected invisible emotional states, the processing unit being trained using a training set comprising HC changes of subjects with known emotional states.

In another aspect, a system for conducting online market research is provided, the system comprising: a server for transmitting computer-readable instructions to a computing device of a participant, the computing device having a display, a network interface coupled to a network, and a camera configured to capture image sequences of a user of the computing device, the computer-readable instructions causing the computing device to simultaneously display at least one content item via the display and capture an image sequence of the participant via the camera, and transmit the captured image sequence to the server via the network interface; and a processing unit configured to process the image sequence to determine a set of bitplanes of a plurality of images in the captured image sequence that represent the hemoglobin concentration (HC) changes of the participant, detect the participant's invisible emotional states based on the HC changes, and output the detected invisible emotional states, the processing unit being trained using a training set comprising HC changes of subjects with known emotional states.

### BRIEF DESCRIPTION OF THE DRAWINGS

The features of the invention will become more apparent in the following detailed description in which reference is made to the appended drawings wherein:

FIG. 1 illustrates a system for conducting online market research and its operating environment in accordance with an embodiment;

FIG. 2 is a schematic diagram of some of the physical components of the server of FIG. 1;

FIG. 3 shows the computing device of FIG. 1 in greater detail;

FIG. 4 is an block diagram of various components of the system for invisible emotion detection of FIG. 1;

FIG. 5 illustrates re-emission of light from skin epidermal and subdermal layers;

FIG. 6 is a set of surface and corresponding transdermal images illustrating change in hemoglobin concentration associated with invisible emotion for a particular human subject at a particular point in time;

FIG. 7 is a plot illustrating hemoglobin concentration changes for the forehead of a subject who experiences positive, negative, and neutral emotional states as a function of time (seconds).

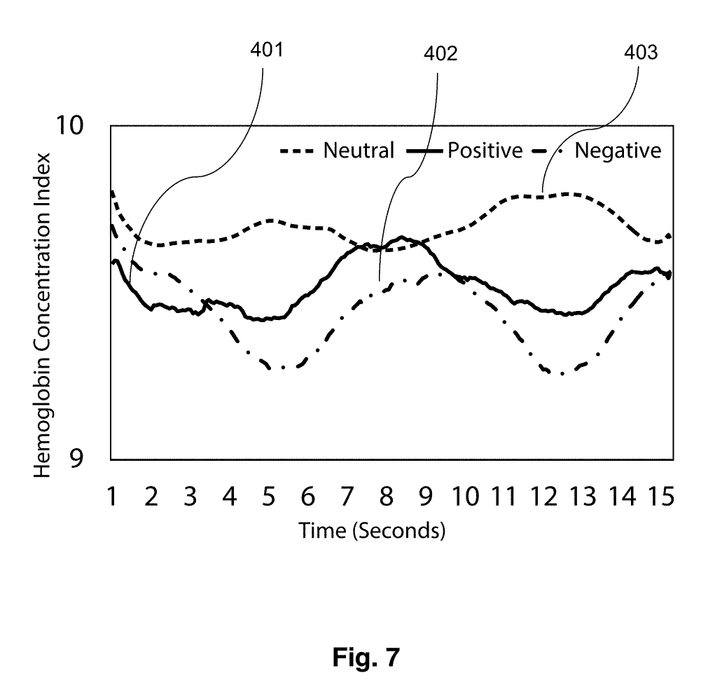

FIG. 8 is a plot illustrating hemoglobin concentration changes for the nose of a subject who experiences positive, negative, and neutral emotional states as a function of time (seconds).

FIG. 9 is a plot illustrating hemoglobin concentration changes for the cheek of a subject who experiences positive, negative, and neutral emotional states as a function of time (seconds).

FIG. 10 is a flowchart illustrating a fully automated transdermal optical imaging and invisible emotion detection system;

FIG. 11 is an illustration of a data-driven machine learning system for optimized hemoglobin image composition;

FIG. 12 is an illustration of a data-driven machine learning system for multidimensional invisible emotion model building;

FIG. 13 is an illustration of an automated invisible emotion detection system;

FIG. 14 is a memory cell; and

FIG. 15 shows the general method of conducting online market research used by the system of FIG. 1.

### DETAILED DESCRIPTION

Embodiments will now be described with reference to the figures. For simplicity and clarity of illustration, where considered appropriate, reference numerals may be repeated among the Figures to indicate corresponding or analogous elements. In addition, numerous specific details are set forth in order to provide a thorough understanding of the embodiments described herein. However, it will be understood by those of ordinary skill in the art that the embodiments described herein may be practiced without these specific details. In other instances, well-known methods, procedures and components have not been described in detail so as not to obscure the embodiments described herein. Also, the description is not to be considered as limiting the scope of the embodiments described herein.

Various terms used throughout the present description may be read and understood as follows, unless the context indicates otherwise: “or” as used throughout is inclusive, as though written “and/or”; singular articles and pronouns as used throughout include their plural forms, and vice versa; similarly, gendered pronouns include their counterpart pronouns so that pronouns should not be understood as limiting anything described herein to use, implementation, performance, etc. by a single gender; “exemplary” should be understood as “illustrative” or “exemplifying” and not necessarily as “preferred” over other embodiments. Further definitions for terms may be set out herein; these may apply to prior and subsequent instances of those terms, as will be understood from a reading of the present description.

Any module, unit, component, server, computer, terminal, engine or device exemplified herein that executes instructions may include or otherwise have access to computer readable media such as storage media, computer storage media, or data storage devices (removable and/or non-removable) such as, for example, magnetic disks, optical disks, or tape. Computer storage media may include volatile and non-volatile, removable and non-removable media implemented in any method or technology for storage of information, such as computer readable instructions, data structures, program modules, or other data. Examples of computer storage media include RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by an application, module, or both. Any such computer storage media may be part of the device or accessible or connectable thereto. Further, unless the context clearly indicates otherwise, any processor or controller set out herein may be implemented as a singular processor or as a plurality of processors. The plurality of processors may be arrayed or distributed, and any processing function referred to herein may be carried out by one or by a plurality of processors, even though a single processor may be exemplified. Any method, application or module herein described may be implemented using computer readable/executable instructions that may be stored or otherwise held by such computer readable media and executed by the one or more processors.

The following relates generally to market research and more specifically to a system and method for conducting online market research. The system permits market research study managers to upload content comprising images, movies, videos, audio, and text related to products, services, advertising, packaging, etc. and select parameters for defining a target group of participants. Registered users satisfying the parameters are invited to participate. Participants may then be selected from the responding invited users. The market research study may be conducted across all participants simultaneously or asynchronously. During the market research study, a participant logs into the computer system via a web browser on their computing device and is presented with the content that is delivered by the computer system. Participants may be prompted to provide feedback via the keyboard or mouse. In addition, image sequences are captured of the participant's face via a camera while participants are viewing the content on the display and sent to the computer system for invisible human emotion detection with a high degree of confidence. The invisible human emotions detected are then used as feedback for the market research study.

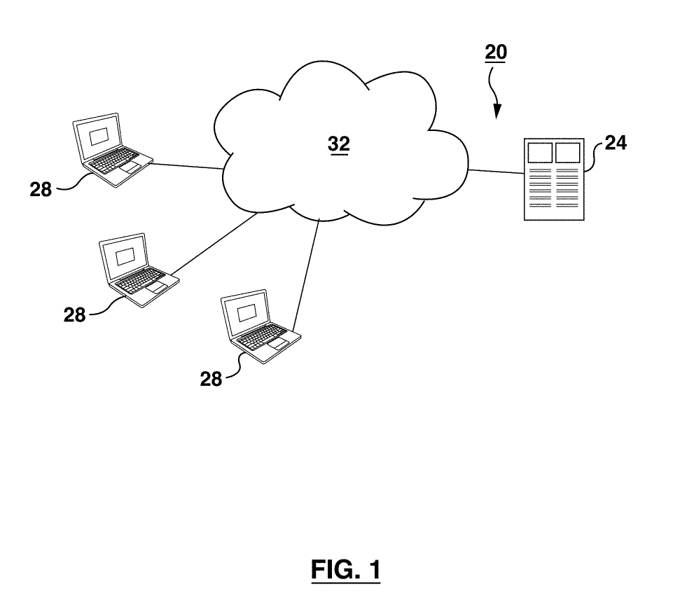

FIG. 1 shows a system 20 for conducting online market research in accordance with an embodiment. A market research server 24 is a computer system that is in communication with a set of computing devices 28 operated by participants in the market research study over a telecommunications network. In the illustrated embodiment, the telecommunications network is the Internet 32. The server 24 can store content in the form of images, videos, audio, and text to be presented to participants. Alternatively, the server 24 can be configured to receive and broadcast a live video and/or audio feed, such as via a video conferencing platform. In some configurations, the content may be broadcast via a separate application and the server 24 can be configured to simply register and process image sequences received from the participants' computing devices 28 to detect invisible human emotions with timing information to map invisible emotions detected with events in content delivered via another platform.

In addition, the server 24 stores trained configuration data enabling it to detect invisible human emotion in image sequences received from the participants' computing devices 28.

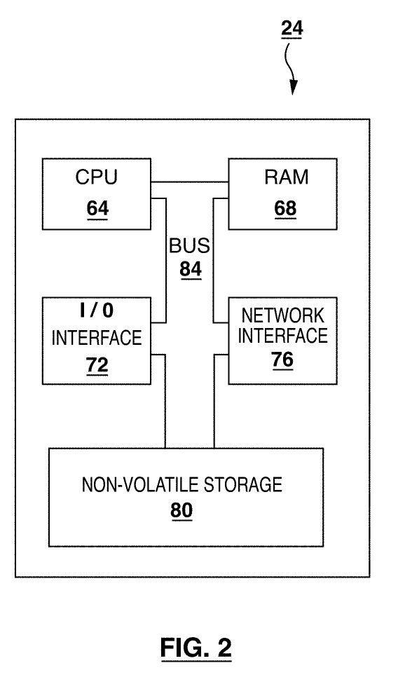

FIG. 2 illustrates a number of physical components of the server 24. As shown, server 24 comprises a central processing unit (“CPU”) 64, random access memory (“RAM”) 68, an input/output (“I/O”) interface 72, a network interface 76, non-volatile storage 80, and a local bus 84 enabling the CPU 64 to communicate with the other components. CPU 64 executes an operating system, a web service, an API, and an emotion detection program. RAM 68 provides relatively responsive volatile storage to the CPU 64. The I/O interface 72 allows for requests to be received from one or more devices, such as a keyboard, a mouse, etc., and outputs information to output devices, such as a display and/or speakers. The network interface 76 permits communication with other systems, such as participants' computing devices 28 and the computing devices of one or more market research study managers. The non-volatile storage 80 stores the operating system and programs, including computer-executable instructions for implementing the web service, the API, and the emotion detection program. During operation of the server 24, the operating system, the programs and the data may be retrieved from the non-volatile storage 80 and placed in the RAM 68 to facilitate execution.

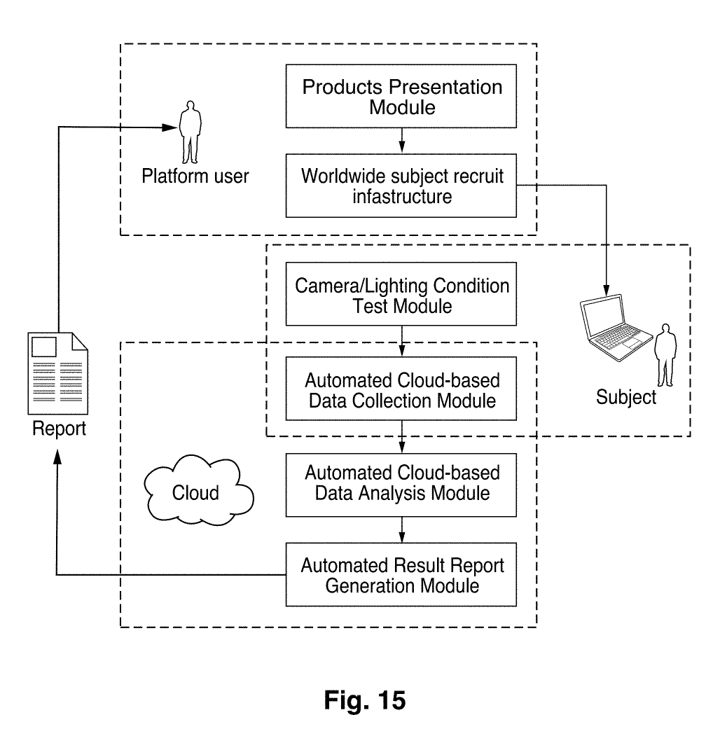

FIG. 15 shows the general method of conducting online market research using the system 20 in one scenario. A products presentation module enables a market research study manager to assemble content in the form of a presentation. A worldwide subject recruitment infrastructure allows for the selection of appropriate candidates for a market research study based on parameters specified by the manager. A camera/lighting condition test module enables the establishment of a baseline for colors captured by the camera 44 of a participant's computing device 28. An automated cloud-based data collection module captures feedback from the computing devices 28 of participants. An automated cloud-based data analysis module analyzes image sequences captured by the camera 44 and other feedback provided by the participant. An automated result report generation module generates a report that is made available to the market research study manager.

A market research study manager seeking to manage a market research study can upload and manage content on the server 24 via the API provided, and select parameters for defining a target group of participants for a market research study. The parameters can include, for example, age, sex, location, income, marital status, number of children, occupation type, etc. Once the content is uploaded, the market research study manager can organize the content in a similar manner to an interactive multimedia slide presentation via a presentation module. Further, the market research study manager can specify when to capture image sequences during presentation of the content to a participant for invisible human emotion detection by the server 24. Where the market research study manager doesn't specify when to capture image sequences, the system 20 is configured to capture image sequences continuously.

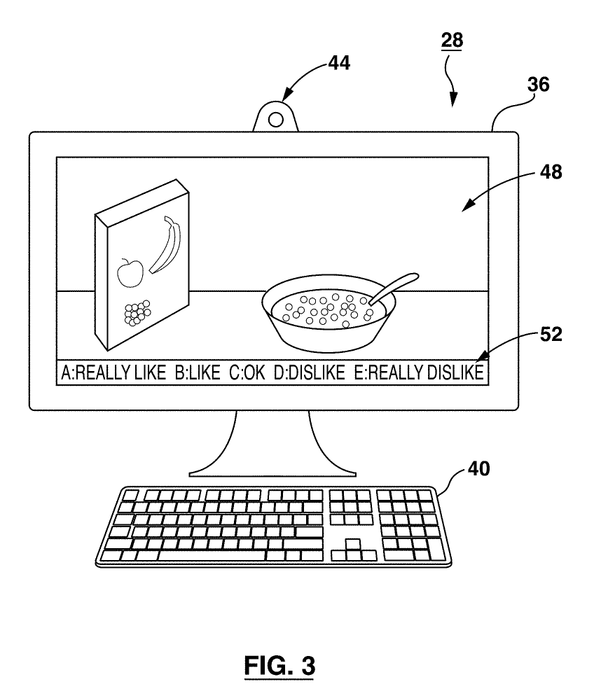

FIG. 3 illustrates an exemplary computing device 28 operated by a participant of a market research study. The computing device 28 has a display 36, a keyboard 40, and a camera 44. The computing device 28 may be in communication with the Internet 32 via any suitable wired or wireless communication type, such as Ethernet, Universal Serial Bus (“USB”), IEEE 802.11 (“Wi-Fi”), Bluetooth, etc. The display 36 presents images, videos, and text associated with a market research study received from the server 24. The camera 44 is configured to capture image sequences of the face (or potentially other body parts) of the participant, and can be any suitable camera type for capturing an image sequence of a consumer's face, such as, for example, a CMOS or CCD camera.

As illustrated, the participant has logged in to the server 24 via a web browser or (other software application) and is participating in a market research study. The content is presented to the participant via the web browser in full screen mode. In particular, an advertisement video is being presented in an upper portion 48 of the display 36. Optionally, text prompting the participant to provide feedback via the keyboard 40 and/or mouse (not shown) is presented in a lower portion 52 of the display 36. Input received from the participant via the keyboard 40 or mouse, as well as image sequences of the participant's face captured by the camera 44, are then sent back to the server 24 for analysis. Timing information is sent with the image sequences to enable understanding of when the image sequences were captured in relation to the content presented.

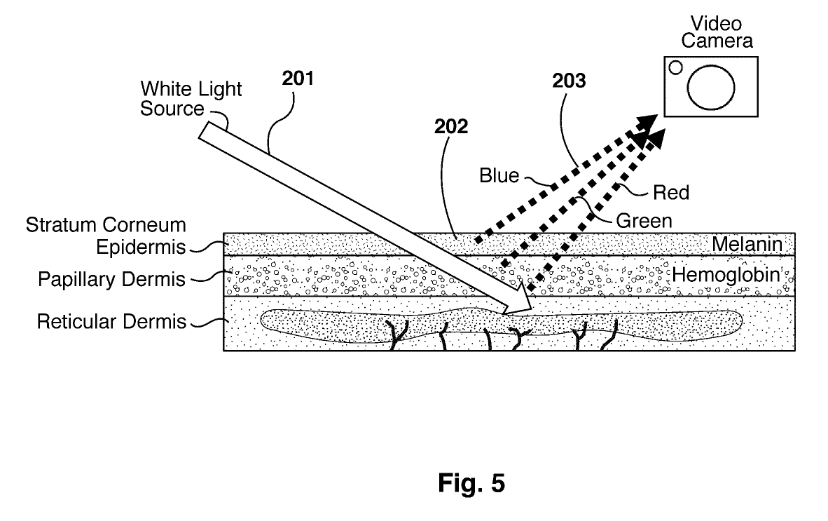

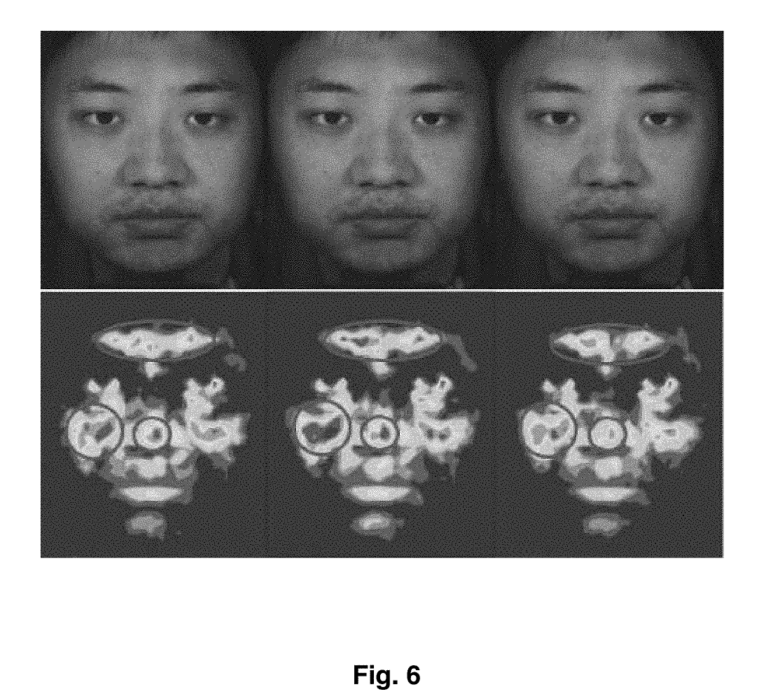

Hemoglobin concentration (HC) can be isolated by the server 24 from raw images taken from the camera 44, and spatial-temporal changes in HC can be correlated to human emotion. Referring now to FIG. 5, a diagram illustrating the re-emission of light from skin is shown. Light (201) travels beneath the skin (202), and re-emits (203) after travelling through different skin tissues. The re-emitted light (203) may then be captured by optical cameras. The dominant chromophores affecting the re-emitted light are melanin and hemoglobin. Since melanin and hemoglobin have different color signatures, it has been found that it is possible to obtain images mainly reflecting HC under the epidermis as shown in FIG. 6.

The system 20 implements a two-step method to generate rules suitable to output an estimated statistical probability that a human subject's emotional state belongs to one of a plurality of emotions, and a normalized intensity measure of such emotional state given a video sequence of any subject. The emotions detectable by the system correspond to those for which the system is trained.

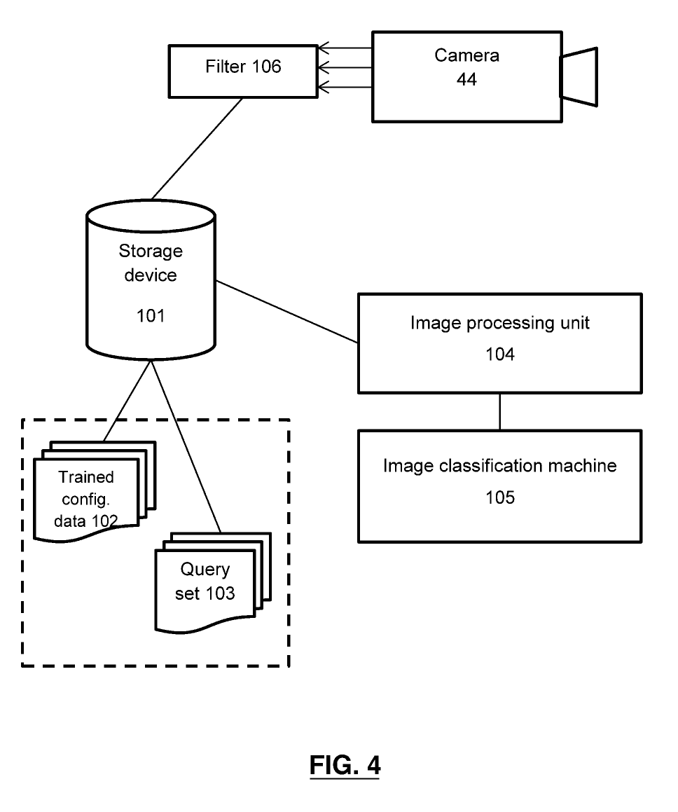

Referring now to FIG. 4, various components of the system 20 configured for invisible emotion detection are shown in isolation. The server 24 comprises an image processing unit 104, an image filter 106, an image classification machine 105, and a storage device 101. A processor of the server 24 retrieves computer-readable instructions from the storage device 101 and executes them to implement the image processing unit 104, the image filter 106, and the image classification machine 105, The image classification machine 105 is configured with training configuration data 102 derived from another computer system trained using a training set of images and is operable to perform classification for a query set of images 103 which are generated from images captured by the camera 44 of the participant's computing device 28, processed by the image filter 106, and stored on the storage device 102.

The sympathetic and parasympathetic nervous systems are responsive to emotion. It has been found that an individual's blood flow is controlled by the sympathetic and parasympathetic nervous system, which is beyond the conscious control of the vast majority of individuals. Thus, an individual's internally experienced emotion can be readily detected by monitoring their blood flow. Internal emotion systems prepare humans to cope with different situations in the environment by adjusting the activations of the autonomic nervous system (ANS); the sympathetic and parasympathetic nervous systems play different roles in emotion regulation with the former regulating up fight-flight reactions whereas the latter serves to regulate down the stress reactions. Basic emotions have distinct ANS signatures. Blood flow in most parts of the face such as eyelids, cheeks and chin is predominantly controlled by the sympathetic vasodilator neurons, whereas blood flowing in the nose and ears is mainly controlled by the sympathetic vasoconstrictor neurons; in contrast, the blood flow in the forehead region is innervated by both sympathetic and parasympathetic vasodilators. Thus, different internal emotional states have differential spatial and temporal activation patterns on the different parts of the face. By obtaining hemoglobin data from the system, facial hemoglobin concentration (HC) changes in various specific facial areas may be extracted. These multidimensional and dynamic arrays of data from an individual are then compared to computational models based on normative data to be discussed in more detail below. From such comparisons, reliable statistically based inferences about an individual's internal emotional states may be made. Because facial hemoglobin activities controlled by the ANS are not readily subject to conscious controls, such activities provide an excellent window into an individual's genuine innermost emotions.

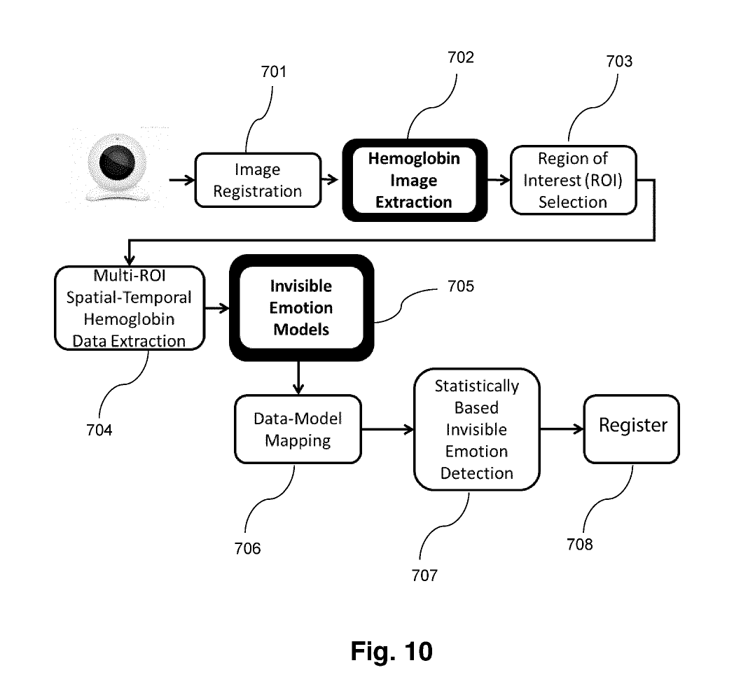

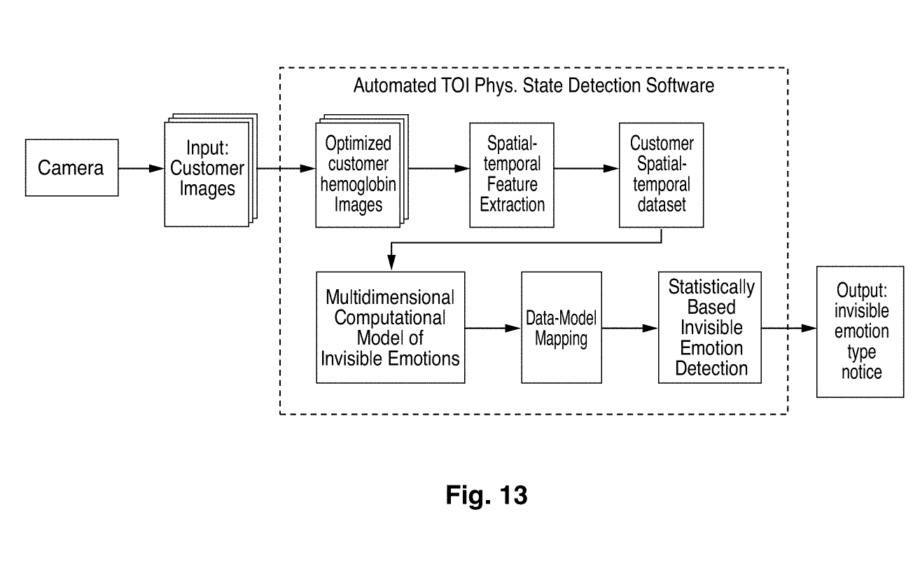

Referring now to FIG. 10, a flowchart illustrating the method of invisible emotion detection performed by the system 20 is shown. The system 20 performs image registration 701 to register the input of a video/image sequence captured of a subject with an unknown emotional state, hemoglobin image extraction 702, ROI selection 703, multi-ROI spatial-temporal hemoglobin data extraction 704, invisible emotion model 705 application, data mapping 706 for mapping the hemoglobin patterns of change, emotion detection 707, and registration 708. FIG. 13 depicts another such illustration of automated invisible emotion detection system.

The image processing unit obtains each captured image or video stream from the camera 44 of the participant's computing device 28 and performs operations upon the image to generate a corresponding optimized HC image of the subject. The image processing unit isolates HC in the captured video sequence. In an exemplary embodiment, the images of the subject's faces are taken at 30 frames per second using the camera 44 of the participant's computing device 28. It will be appreciated that this process may be performed with various types of digital cameras and lighting conditions.

Isolating HC is accomplished by analyzing bitplanes in the video sequence to determine and isolate a set of the bitplanes that provide high signal to noise ratio (SNR) and, therefore, optimize signal differentiation between different emotional states on the facial epidermis (or any part of the human epidermis). The determination of high SNR bitplanes is made with reference to a first training set of images constituting the captured video sequence, coupled with EKG, pneumatic respiration, blood pressure, laser Doppler data from the human subjects from which the training set is obtained. The EKG and pneumatic respiration data are used to remove cardiac, respiratory, and blood pressure data in the HC data to prevent such activities from masking the more-subtle emotion-related signals in the HC data. The second step comprises training a machine to build a computational model for a particular emotion using spatial-temporal signal patterns of epidermal HC changes in regions of interest (“ROIs”) extracted from the optimized “bitplaned” images of a large sample of human subjects.

For training, video images of test subjects exposed to stimuli known to elicit specific emotional responses are captured. Responses may be grouped broadly (neutral, positive, negative) or more specifically (distressed, happy, anxious, sad, frustrated, intrigued, joy, disgust, angry, surprised, contempt, etc.). In further embodiments, levels within each emotional state may be captured. Preferably, subjects are instructed not to express any emotions on the face so that the emotional reactions measured are invisible emotions and isolated to changes in HC. To ensure subjects do not “leak” emotions in facial expressions, the surface image sequences may be analyzed with a facial emotional expression detection program. EKG, pneumatic respiratory, blood pressure, and laser Doppler data may further be collected using an EKG machine, a pneumatic respiration machine, a continuous blood pressure machine, and a laser Doppler machine and provides additional information to reduce noise from the bitplane analysis, as follows.

ROIs for emotional detection (e.g., forehead, nose, and cheeks) are defined manually or automatically for the video images. These ROIs are preferably selected on the basis of knowledge in the art in respect of ROIs for which HC is particularly indicative of emotional state. Using the native images that consist of all bitplanes of all three R, G, B channels, signals that change over a particular time period (e.g., 10 seconds) on each of the ROIs in a particular emotional state (e.g., positive) are extracted. The process may be repeated with other emotional states (e.g., negative or neutral). The EKG and pneumatic respiration data may be used to filter out the cardiac, respirator, and blood pressure signals on the image sequences to prevent non-emotional systemic HC signals from masking true emotion-related HC signals. Fast Fourier transformation (FFT) may be used on the EKG, respiration, and blood pressure data to obtain the peek frequencies of EKG, respiration, and blood pressure, and then notch filers may be used to remove HC activities on the ROIs with temporal frequencies centering around these frequencies. Independent component analysis (ICA) may be used to accomplish the same goal.

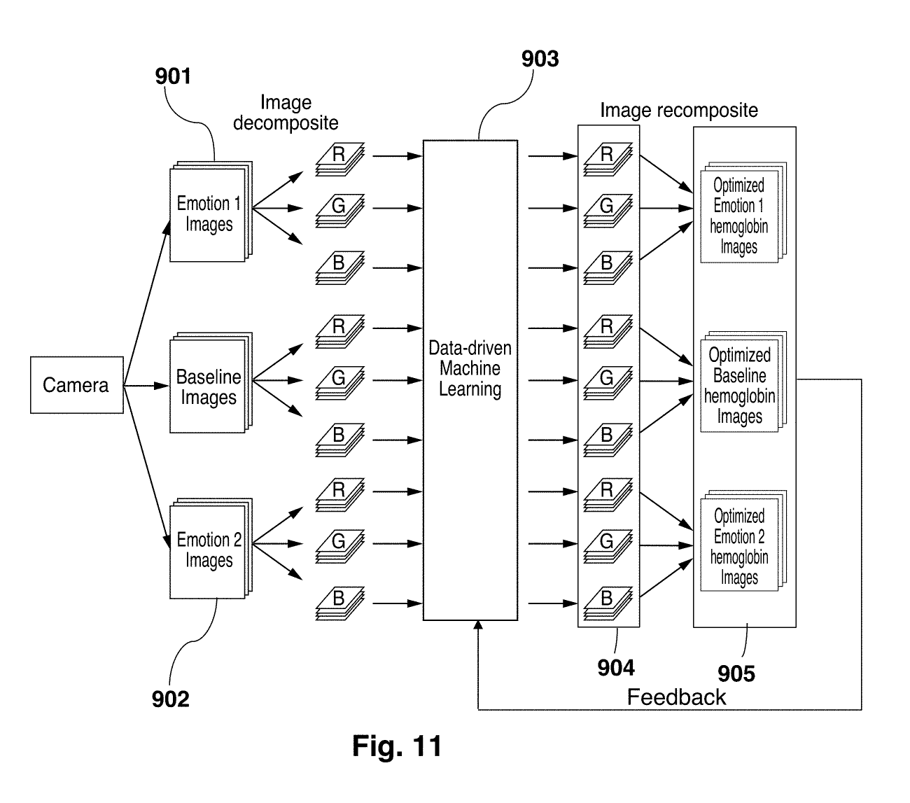

Referring now to FIG. 11 an illustration of data-driven machine learning for optimized hemoglobin image composition is shown. Using the filtered signals from the ROIs of two or more than two emotional states 901 and 902, machine learning 903 is employed to systematically identify bitplanes 904 that will significantly increase the signal differentiation between the different emotional state and bitplanes that will contribute nothing or decrease the signal differentiation between different emotional states. After discarding the latter, the remaining bitplane images 905 that optimally differentiate the emotional states of interest are obtained. To further improve SNR, the result can be fed back to the machine learning 903 process repeatedly until the SNR reaches an optimal asymptote.

The machine learning process involves manipulating the bitplane vectors (e.g., 8×8×8, 16×16×16) using image subtraction and addition to maximize the signal differences in all ROIs between different emotional states over the time period for a portion (e.g., 70%, 80%, 90%) of the subject data and validate on the remaining subject data. The addition or subtraction is performed in a pixel-wise manner. An existing machine learning algorithm, the Long Short Term Memory (LSTM) neural network, or a suitable alternative (e.g., deep learning) thereto is used to efficiently and obtain information about the improvement of differentiation between emotional states in terms of accuracy, which bitplane(s) contributes the best information, and which does not in terms of feature selection. The Long Short Term Memory (LSTM) neural network or a suitable alternative allows us to perform group feature selections and classifications. The LSTM machine learning algorithm is discussed in more detail below. From this process, the set of bitplanes to be isolated from image sequences to reflect temporal changes in HC is obtained. An image filter is configured to isolate the identified bitplanes in subsequent steps described below.

The image classification machine 105 is configured with trained configuration data 102 from a training computer system previously trained with a training set of images captured using the above approach. In this manner, the image classification machine 105 benefits from the training performed by the training computer system. The image classification machine 104 classifies the captured image as corresponding to an emotional state. In the second step, using a new training set of subject emotional data derived from the optimized bitplane images provided above, machine learning is employed again to build computational models for emotional states of interests (e.g., positive, negative, and neural).

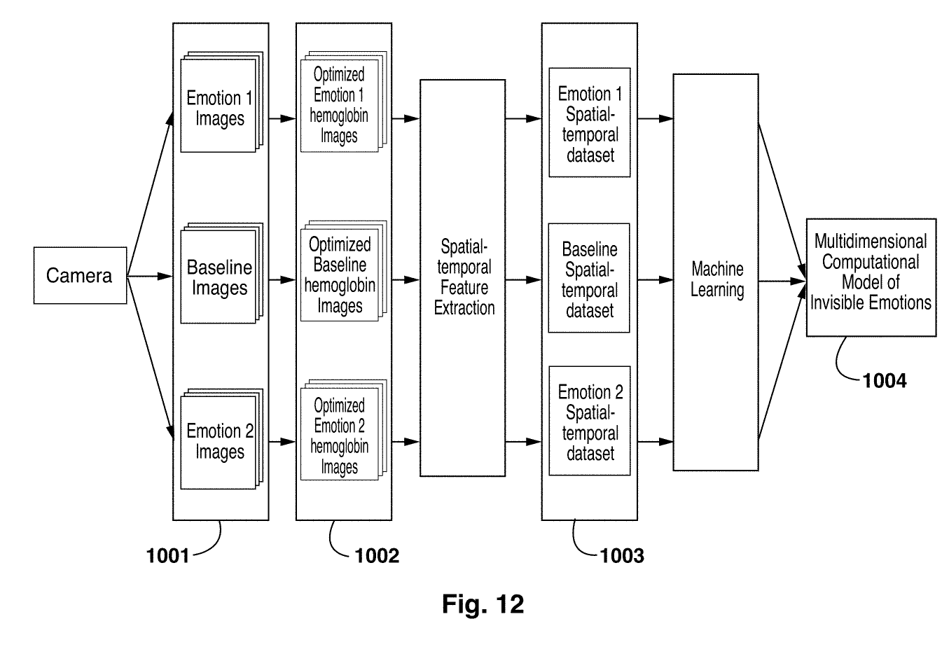

Referring now to FIG. 12, an illustration of data-driven machine learning for multidimensional invisible emotion model building is shown. To create such models, a second set of training subjects (preferably, a new multi-ethnic group of training subjects with different skin types) is recruited, and image sequences 1001 are obtained when they are exposed to stimuli eliciting known emotional response (e.g., positive, negative, neutral). An exemplary set of stimuli is the International Affective Picture System, which has been commonly used to induce emotions and other well established emotion-evoking paradigms. The image filter is applied to the image sequences 1001 to generate high HC SNR image sequences. The stimuli could further comprise non-visual aspects, such as auditory, taste, smell, touch or other sensory stimuli, or combinations thereof.

Using this new training set of subject emotional data 1003 derived from the bitplane filtered images 1002, machine learning is used again to build computational models for emotional states of interests (e.g., positive, negative, and neural) 1003. Note that the emotional state of interest used to identify remaining bitplane filtered images that optimally differentiate the emotional states of interest and the state used to build computational models for emotional states of interests must be the same. For different emotional states of interests, the former must be repeated before the latter commences.

The machine learning process again involves a portion of the subject data (e.g., 70%, 80%, 90% of the subject data) and uses the remaining subject data to validate the model. This second machine learning process thus produces separate multidimensional (spatial and temporal) computational models of trained emotions 1004.

To build different emotional models, facial HC change data on each pixel of each subject's face image is extracted (from Step 1) as a function of time when the subject is viewing a particular emotion-evoking stimulus. To increase SNR, the subject's face is divided into a plurality of ROIs according to their differential underlying ANS regulatory mechanisms mentioned above, and the data in each ROI is averaged.

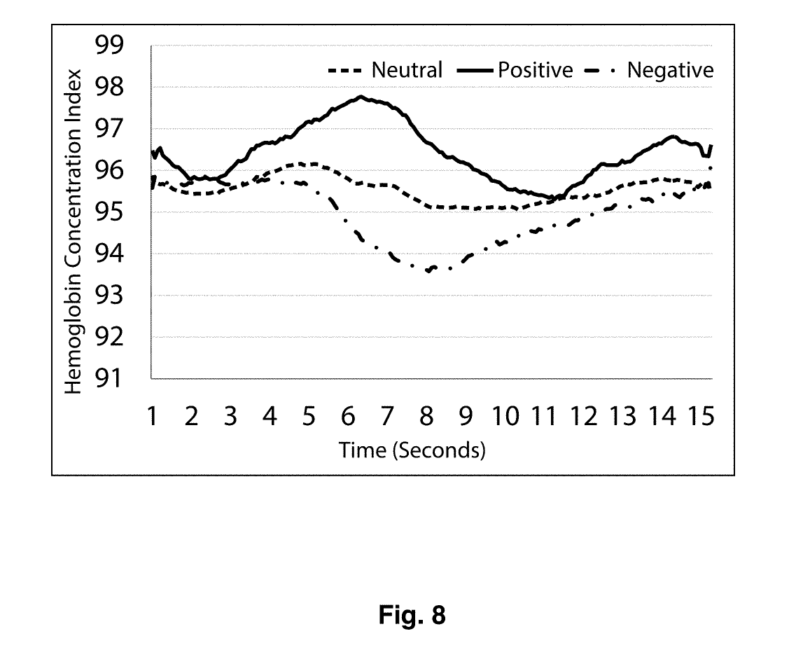

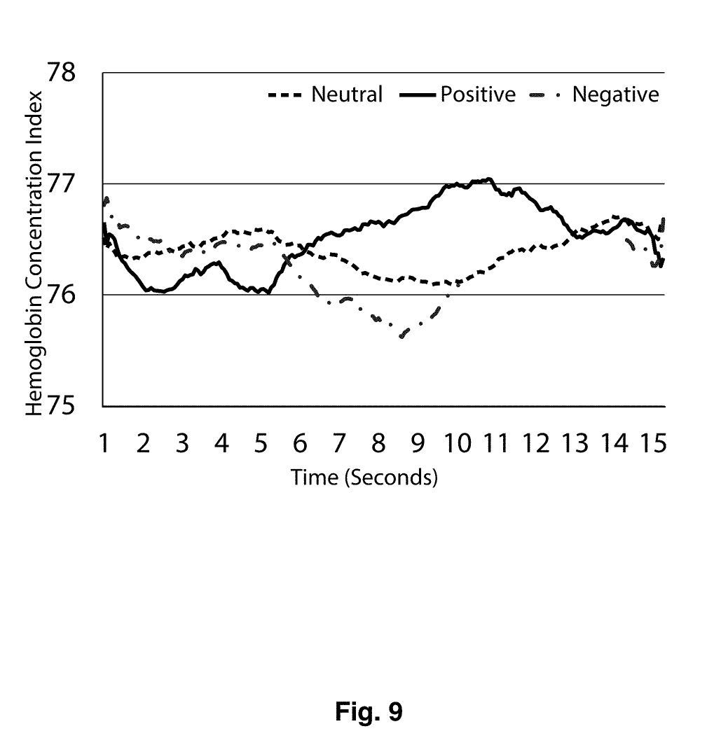

Referring now to FIG. 4, a plot illustrating differences in hemoglobin distribution for the forehead of a subject is shown. Though neither human nor computer-based facial expression detection system may detect any facial expression differences, transdermal images show a marked difference in hemoglobin distribution between positive 401, negative 402 and neutral 403 conditions. Differences in hemoglobin distribution for the nose and cheek of a subject may be seen in FIG. 8 and FIG. 9 respectively.

The Long Short Term Memory (LSTM) neural network, or a suitable alternative such as non-linear Support Vector Machine, and deep learning may again be used to assess the existence of common spatial-temporal patterns of hemoglobin changes across subjects. The Long Short Term Memory (LSTM) neural network or an alternative is trained on the transdermal data from a portion of the subjects (e.g., 70%, 80%, 90%) to obtain a multi-dimensional computational model for each of the three invisible emotional categories. The models are then tested on the data from the remaining training subjects.

These models form the basis for the trained configuration data 102.

Following these steps, it is now possible to obtain image sequences of the participant's face captured by the camera 44 and received by the server 24, and apply the HC extracted from the selected bitplanes to the computational models for emotional states of interest. The output will be a notification corresponding to (1) an estimated statistical probability that the subject's emotional state belongs to one of the trained emotions, and (2) a normalized intensity measure of such emotional state. For long running video streams when emotional states change and intensity fluctuates, changes of the probability estimation and intensity scores over time relying on HC data based on a moving time window (e.g., 10 seconds) may be reported. It will be appreciated that the confidence level of categorization may be less than 100%.

Two example implementations for (1) obtaining information about the improvement of differentiation between emotional states in terms of accuracy, (2) identifying which bitplane contributes the best information and which does not in terms of feature selection, and (3) assessing the existence of common spatial-temporal patterns of hemoglobin changes across subjects will now be described in more detail. One such implementation is a recurrent neural network.

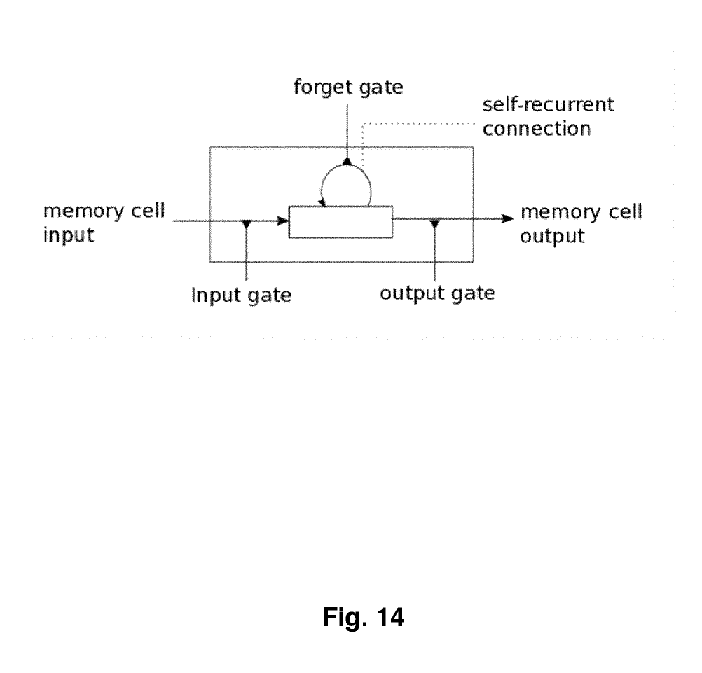

One recurrent neural network is known as the Long Short Term Memory (LSTM) neural network, which is a category of neural network model specified for sequential data analysis and prediction. The LSTM neural network comprises at least three layers of cells. The first layer is an input layer, which accepts the input data. The second (and perhaps additional) layer is a hidden layer, which is composed of memory cells (see FIG. 14). The final layer is output layer, which generates the output value based on the hidden layer using Logistic Regression.

Each memory cell, as illustrated, comprises four main elements: an input gate, a neuron with a self-recurrent connection (a connection to itself), a forget gate and an output gate. The self-recurrent connection has a weight of 1.0 and ensures that, barring any outside interference, the state of a memory cell can remain constant from one time step to another. The gates serve to modulate the interactions between the memory cell itself and its environment. The input gate permits or prevents an incoming signal to alter the state of the memory cell. On the other hand, the output gate can permit or prevent the state of the memory cell to have an effect on other neurons. Finally, the forget gate can modulate the memory cell's self-recurrent connection, permitting the cell to remember or forget its previous state, as needed.

The equations below describe how a layer of memory cells is updated at every time step t. In these equations:

xₜ is the input array to the memory cell layer at time t. In our application, this is the blood flow signal at all ROIs

$${\vec{x}}_{t} = {[ \begin{matrix}{x}_{1 t} & {x}_{2 t} & K & {x}_{nt}\end{matrix} ]}^{′}$$

Wᵢ, W_{f}, W_{c}, Wₒ, Uᵢ, U_{f}, U_{c}, U₀ and Vₒ are weights matrices; and
bᵢ, b_{f}, b_{c} and bₒ are bias vectors

First, we compute the values for iₜ, the input gate, and Cₜ^{%} the candidate value for the states of the memory cells at time t:

iₜ=σ(Wᵢxₜ+Uᵢhₜ₋₁+bᵢ)

Cₜ^{%}=tan h(W_{c}xₜ+U_{c}hₜ₋₁+b_{c})

Second, we compute the value for fₜ, the activation of the memory cells' forget gates at time t:

fₜ=σ(W_{f}xₜ+U_{f}hₜ₋₁+b_{f})

Given the value of the input gate activation iₜ, the forget gate activation fₜ and the candidate state value Cₜ^{%}, we can compute Cₜ the memory cells' new state at time t:

Cₜ=iₜ*Cₜ^{%}+fₜ*Cₜ₋₁

With the new state of the memory cells, we can compute the value of their output gates and, subsequently, their outputs:

σₜ=σ(Wₒxₜ+Uₒhₜ₋₁+VₒCₜ+bₒ)

hₜ=0ₜ*tan h(Cₜ)

Based on the model of memory cells, for the blood flow distribution at each time step, we can calculate the output from memory cells. Thus, from an input sequence x₀, x₁, x₂, L, xₙ, the memory cells in the LSTM layer will produce a representation sequence h₀, h₁, h₂, L, hₙ.

The goal is to classify the sequence into different conditions. The Logistic Regression output layer generates the probability of each condition based on the representation sequence from the LSTM hidden layer. The vector of the probabilities at time step t can be calculated by:

pₜ=softmax(Wₒᵤₜₚᵤₜ+hₜ+bₒᵤₜₚᵤₜ)

where Wₒᵤₜₚᵤₜ is the weight matrix from the hidden layer to the output layer, and bₒᵤₜₚᵤₜ is the bias vector of the output layer. The condition with the maximum accumulated probability will be the predicted condition of this sequence.

The server 24 registers the image streams captured by the camera 44 and received from the participant's computing device 28, and makes a determination of the invisible emotion detected using the process described above. An intensity of the invisible emotion detected is also registered. The server 24 then correlates the detected invisible emotions detected to particular portions of the content using the timing information received from the participant's computing device 28, as well as the other feedback received from the participant via the keyboard and mouse of the participant's computing device 28. This feedback can then be summarized by the server 24 and made available to the market research study manager for analysis.

The server 24 can be configured to discard the image sequences upon detecting the invisible emotion and registering their timing relative to the content.

In another embodiment, the server 24 can perform gaze-tracking to identify what part of the display in particular the participant is looking at when an invisible human emotion is detected. In order to improve the gaze-tracking, a calibration can be performed by presenting the participant with icons or other images at set locations on the display and directing the participant to look at them, or simply at the corners or edges of the display, while capturing images of the participant's eyes. In this manner, the server 24 can learn the size and position of the display that a participant is using and then use this information to determine what part of the display the participant is looking at during the presentation of content on the display to determine to identify what the participant is reacting to when an invisible human emotion is detected.

In different embodiments, as part of the registration process, the above-described approach for generating trained configuration data can be executed using only image sequences for the particular user. The user can be shown particular videos, images, etc. that are highly probable to trigger certain emotions, and image sequences can be captured and analyzed to generate the trained configuration data. In this manner, the trained configuration data can also take into consideration the lighting conditions and color characteristics of the user's camera.

Although the invention has been described with reference to certain specific embodiments, various modifications thereof will be apparent to those skilled in the art without departing from the spirit and scope of the invention as outlined in the claims appended hereto. The entire disclosures of all references recited above are incorporated herein by reference.

We claim:

1. A method for conducting online market research, the method comprising:
transmitting computer-readable instructions to a computing device of a participant, the computing device having a display, a network interface coupled to a network, and a camera configured to capture image sequences of a user of the computing device, the computer-readable instructions causing the computing device to simultaneously display at least one content item via the display and capture an image sequence of the participant via the camera, and transmit the captured image sequence to a server via the network interface; and
processing the image sequence using a processing unit configured to determine a set of bitplanes of a plurality of images in the captured image sequence that represent the hemoglobin concentration (HC) changes of the participant, detect the participant's invisible emotional states based on the HC changes, and output the detected invisible emotional states, the processing unit being trained using a training set comprising HC changes of subjects with known emotional states.

2. The method of claim 1, wherein the detecting the person's invisible emotional states based on HC changes comprises generating an estimated statistical probability that the person's emotional state conforms to a known emotional state from the training set, and a normalized intensity measure of such determined emotional state.

3. The method of claim 1, wherein the computer-readable instructions further cause the computing device to transmit timing information relating to timing of display of the at least one content item.

4. The method of claim 3, further comprising correlating the detected invisible emotional states to particular portions of the content using the timing information received from the participant's computing device.

5. The method of claim 4, further comprising performing, by the processing unit, gaze tracking to identify what part of the display in particular the participant was looking at when a particular invisible emotional state was detected, to determine whether the participant was looking at the at least one content item during the occurrence of the detected invisible human emotion.

6. The method of claim 5, wherein the computer-readable instructions further cause the computing device to test a camera/lighting condition of the camera for calibrating the camera for gaze tracking.

7. The method of claim 1, further comprising, selecting, by the processing unit, the participant based on a set of received parameters.

8. The method of claim 7, wherein the parameters comprise any one of age, sex, location, income, marital status, number of children, or occupation type.

9. The method of claim 1, wherein the at least one content item comprises at least one of an image, a video or text.

10. The method of claim 1, further comprising receiving an input for specifying selective capture of image sequences.

11. A system for conducting online market research, the system comprising:
a server for transmitting computer-readable instructions to a computing device of a participant, the computing device having a display, a network interface coupled to a network, and a camera configured to capture image sequences of a user of the computing device, the computer-readable instructions causing the computing device to simultaneously display at least one content item via the display and capture an image sequence of the participant via the camera, and transmit the captured image sequence to the server via the network interface; and
a processing unit configured to process the image sequence to determine a set of bitplanes of a plurality of images in the captured image sequence that represent the hemoglobin concentration (HC) changes of the participant, detect the participant's invisible emotional states based on the HC changes, and output the detected invisible emotional states, the processing unit being trained using a training set comprising HC changes of subjects with known emotional states.

12. The system of claim 10, wherein the detecting the person's invisible emotional states based on HC changes comprises generating an estimated statistical probability that the person's emotional state conforms to a known emotional state from the training set, and a normalized intensity measure of such determined emotional state.

13. The system of claim 10, wherein the computer-readable instructions further cause the computing device to transmit timing information relating to timing of display of the at least one content item.

14. The system of claim 13, wherein the processing unit is further configured to correlate the detected invisible emotional states to particular portions of the content using the timing information received from the participant's computing device.

15. The system of claim 14, wherein the processing unit is further configured to perform gaze tracking to identify what part of the display in particular the participant was looking at when a particular invisible emotional state was detected, to determine whether the participant was looking at the at least one content item during the occurrence of the detected invisible human emotion.

16. The system of claim 15, wherein the computer-readable instructions further cause the computing device to test a camera/lighting condition of the camera for calibrating the camera for gaze tracking.

17. The system of claim 10, wherein the processing unit is further configured to select the participant based on a set of received parameters.

18. The system of claim 17, wherein the parameters comprise any one of age, sex, location, income, marital status, number of children, or occupation type.

19. The system of claim 10, wherein the at least one content item comprises at least one of an image, a video or text.

20. The system of claim 10, wherein the server is further configured to receive an input for specifying selective capture of image sequences.
